(19) United States
(12) Patent Application Publication
(10) Pub. No.: US 2010/0222701 A1
(43) Pub. Date: Sep. 2, 2010

(54) BODY LIQUID COLLECTING AUXILIARY MEMBER AND BODY LIQUID COLLECTING DEVICE USING THE AUXILIARY MEMBER

(75) Inventors: Soh Ohzawa, Toyonaka-shi (JP); Naoki Izumiya, Hidaka-shi (JP); Masaaki Takama, Hino-shi (JP); Kenji Konno, Sakai-shi (JP)

Correspondence Address: SIDLEY AUSTIN LLP, 717 NORTH HARWOOD, SUITE 3400, DALLAS, TX, 75201, US

(73) Assignee: Konica Minolta Opto Inc., Hachioji-shi Toyko (JP)

(21) Appl. No.: 12/668,959

(22) PCT Filed: Jun. 27, 2008
(86) PCT No.: PCT/JP2008/061719

§ 371 Date: Jan. 13, 2010

(30) Foreign Application Priority Data
Jul. 17, 2007 (JP) 2007-185976

(51) Int. Cl. A61B 5/15

(52) U.S. Cl. 600/573

(57) ABSTRACT

Provided is a body liquid collecting device comprising a body liquid collecting auxiliary sheet (9), and a transparent member (12) made integral with the body liquid collecting auxiliary sheet (9) and having a lens (11) for collecting laser beam from a semiconductor laser (4b) such that it is interposed between the body liquid collecting auxiliary sheet (9) and the lens (11), so that the surface of the auxiliary sheet (9) may have its surface positioned at the focal point of the lens (11). As a result, the laser beam can be collected at a predetermined portion (or the focal point portion), so that a micro-hole can be reliably formed with a relatively low laser power.

### TECHNICAL FIELD

The present invention relates to a body fluid collection auxiliary sheet to be used on the occasion of collecting body fluid such as intercellular substance liquid and blood by forming a minute hole in a living body surface by applying laser light thereto, and a body fluid collection device using the body fluid collection auxiliary sheet.

### BACKGROUND ART

As an example of the method for collecting the body fluid from a living body, blood collecting by using a hypodermic needle is most usually performed. However, such method causes problems, for example, the method may give uneasiness and pain to the patient and infection may be caused by bacteria existing on the hypodermic needle. Moreover, when collecting is continuously carried out, for example, in the case of monitoring glucose concentration in blood (blood sugar level) every 30 minutes or 1 hour for a follow-up after surgical operation, it is necessary to stick a hypodermic needle into the body for every monitoring, so that patients may bear a big burden with their skin losing elasticity.

Therefore, a method is recently gathering attention, in which method intercellular substance liquid, instead of blood, is collected to reduce the burden to the patient. In this method, a minute hole is made in a skin surface but not reaching to the corium where blood vessels run. This method gets attention for the following reasons: Intercellular substance liquid is used as alternatives of blood for check because the intercellular substance liquid oozing out from the minute hole has almost the same ingredients as the blood except that blood corpuscles are not contained, and no or only little pain is caused at the time of making the minute hole. A low invasive method using laser light is applied to make a minute hole to reduce the possibility of infection through a hypodermic needle and to reduce uneasiness of patient caused by a process where the body is stuck with a pointed object.

For example, a method for conversing laser light at the living body surface is disclosed in Patent Document 1. Patent Document 2 also discloses a method for making a minute hole, in which method included is a device, for measuring by using a censor, having a portion for making a minute hole through which laser light is introduced.

However, the light absorption coefficient of skin largely differs depending to interracial and interpersonal differences, and the light absorption coefficient is low for some wavelength. Consequently, Patent Document 3 discloses a method in which a body fluid collection auxiliary sheet for absorbing and converting light into heat is placed on a living body surface and light is focused on the sheet so as to transfer the light energy to the living body surface with high efficiency to form a minute hole.

Patent Document 1: Japanese Laid-open Patent Application Publication No. 2004-195245

Patent Document 2: Japanese Translation of PCT Application International Application Publication No. 2002-503118

Patent Document 3: U.S. Pat. No. 6,530,915

### DISCLOSURE OF INVENTION

### Problems to be Solved by the Invention

It is difficult, by the above known technology, to precisely converge laser light onto a body fluid collection auxiliary sheet contacted with the living body surface so that greater light energy is necessary to make a minute hole and it is difficult to surely make a minute hole.

An object of the invention is to provide a body fluid collection auxiliary sheet in which laser light is precisely converged so that the minute hole can be surely formed, and to provide a body fluid collection device using the body fluid collection auxiliary sheet.

### Means for Solving the Object

Problems of the invention will be solved by the following configurations.

Item 1. An auxiliary sheet for collecting body fluid which is used when collecting body fluid through a minute hole formed by applying laser light to a living body surface, and is attached to the living body surface to contribute to forming the minute hole, the auxiliary sheet, comprising:

a light convergence member provided, on the other side of a side which is stuck to the living body surface, to converge laser light applied.

Item 2. The auxiliary sheet for collecting body fluid of Item 1, comprising:

a photothermal convergence function for absorbing the laser light and converting the laser light into heat,

wherein the minute hole is made by the converted heat.

Item 3. The auxiliary sheet for collecting body fluid of Item 1 or 2, wherein the light convergence member is a lens, and a transparent member is further provided between the lens and auxiliary sheet so that the surface of the auxiliary sheet for collecting body fluid is positioned at a focal point of the lens.

Item 4. The auxiliary sheet for collecting body fluid of Item 3, wherein a plurality of the lenses are arranged in an array, and a plurality of the minute holes are able to be simultaneously made.

Item 5. The auxiliary sheet for collecting body fluid of Item 3 or 4, wherein the lens is a graded index lens, and the thickness of the graded index lens is adjusted such that the surface of the auxiliary sheet is positioned at a focal point of the graded index lens.

Item 6. The auxiliary sheet for collecting body fluid of Item 3 or 4, wherein the following conditional relationship is satisfied:

|(f−n·t)/Fn|≦0.2

where:

f (mm) is a focal length of the lens in air;

t (mm) is the thickness of the transparent member;

n is a refractive index of the transparent member; and

Fn is an open F number of the lens.

Item 7. The auxiliary sheet for collecting body fluid of Item 2 or 3, wherein the following conditional relationship is satisfied:

0.01<L²/(Fn·E·t³)<0.1

where:

2·L (mm) is a distance between supporting points when pressed against the living body surface;

Fn is an open F number of the lens;

t is a thickness;

E is a Young's modulus.

Item 8. A body fluid collection device, comprising:

a chamber for defining a space for collecting body fluid, which chamber has an opened bottom surface and top surface having an entrance opening for the laser light to enter, and is disposed in contact with the living body surface on which the auxiliary sheet for collecting body fluid of any one of Items 1 to 7 is stuck; and

a laser generation device provided on the chamber, the laser generation device including:
a semiconductor laser; and
a collimator lens for converting the laser light from the semiconductor laser into a parallel light having a predetermined beam diameter, and for supplying the laser light to the light convergence member through the entrance opening.

Item 9. The body fluid collection device of Item 8, further comprising:

a suction member for reducing a pressure in the chamber and for suctioning body fluid which has oozed out.

### ADVANTAGE OF THE INVENTION

1. An auxiliary sheet for collecting body fluid which is used when collecting body fluid through a minute hole formed by applying laser light to a living body surface, and is attached to the living body surface to contribute to forming the minute hole, the auxiliary sheet, comprising:

a light convergence member provided, on the other side of a side which is stuck to the living body surface, to converge laser light applied.

According to the invention, a light convergence member is provided on one side, of the body fluid collection auxiliary sheet, that is opposite to the other side that contacts with the living body surface, and the body fluid collection auxiliary sheet contribute to forming a minute hole in a living body surface when used in the process where the minute hole is made by applying laser light to the living body surface so that body fluid, such as intercellular substance liquid and blood, oozing through the minute hole is collected.

This arrangement provides a body fluid collection sheet in which laser light is precisely converged at the specific point of the body fluid collection auxiliary sheet (the focal point of the light convergence member) so that a minute hole is surely formed by relatively low laser power, and a body fluid collection device using the body fluid collection sheet.

### BRIEF DESCRIPTION OF THE DRAWINGS

FIG. 1 shows a constitution of a body fluid collection device according to an embodiment of the invention;

FIG. 2 shows an enlarged cross sectional view of a stacked structure of a body fluid collection auxiliary sheet according to an embodiment of the invention.

FIG. 3 shows an example of the shape when the light convergence surface of the lens stacked on the body fluid collection auxiliary sheet is made to be Fresnel shape;

FIG. 4 shows an example of the shape when he light convergence surface of the lens stacked on the body fluid collection auxiliary sheet is made to be aspherical;

FIG. 5 shows a simulation result of the shapes of the beam spot with respect to a defocus amount when the Fresnel lens is used;

FIG. 6 shows a simulation result of the shape of the beam spot with respect to a deviation of focal point when the aspherical lens is used;

FIG. 7 is a diagram illustrating the influence of positional deviation of the laser generation device with respect to the chamber;

FIG. 8 is a diagram illustrating the influence of positional deviation of the laser generation device with respect to the chamber;

FIG. 9 shows an enlarged cross sectional view of the stacked structure of a body fluid collection auxiliary sheet according to another embodiment of the invention;

FIG. 10 shows an example of the shape when the light convergence member stacked on the body fluid collection auxiliary sheet is made of a GRIN lens;

FIG. 11 shows a simulation result of the shape of the beam spot with respect to a deviation of focal point when the GRIN lens is used;

FIG. 12 shows a modeled deformation of a planar plate when force is applied.

### NUMERALS

1: Body fluid collection device
2: Living body surface
3: Chamber
3a: Entrance opening
4: Laser generation apparatus
4b: Semiconductor laser
4c: Collimator lens
4d: Driving circuit board
4e: Case
5: Liquid transfer tube
6: Pressure reduction device
7: Sensor
8: Body fluid collection receptacle
9: Body fluid collection auxiliary sheet
11: Lens
12: Transparent member

### BEST MODE FOR CARRYING OUT THE INVENTION

### Embodiment 1

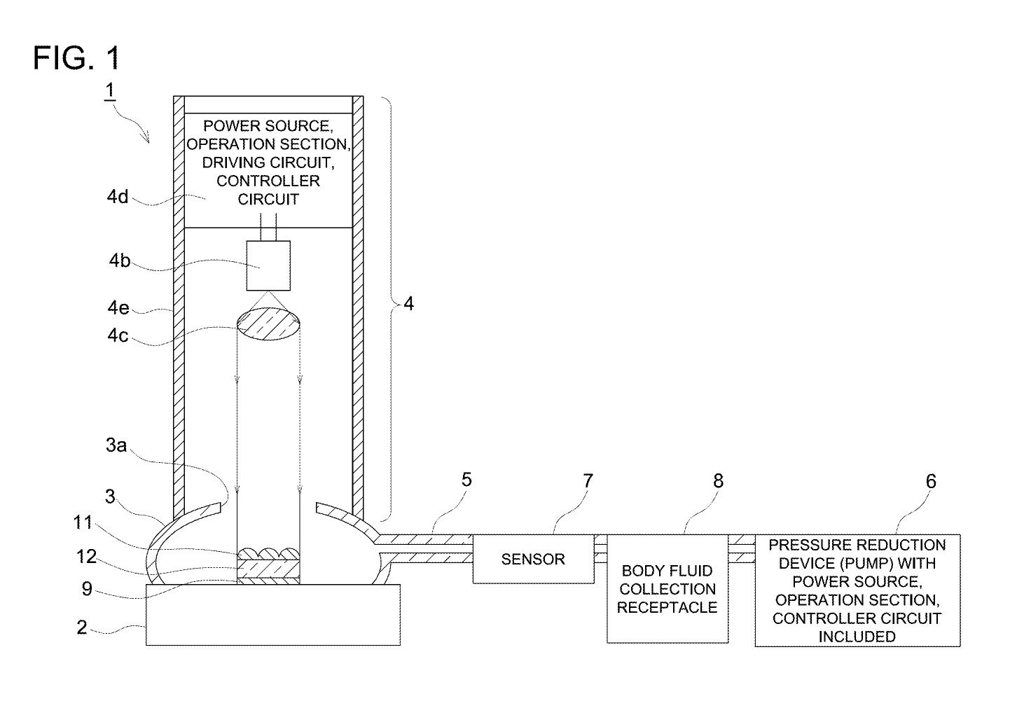

FIG. 1 shows a configuration of a body fluid collection device according to an embodiment of the invention. This body fluid collection device is used for monitoring the glucose concentration (blood glucose level) in the blood of a patient after operation by collecting the intercellular substance liquid at a predetermined interval such as 30 minutes or 1 hour. Other than that, it is also usable for measuring alcohol concentration.

The body fluid collection device 1 is approximately constituted of a chamber 3 with a bottom, having an opening therein, to be contacted with the living body surface such as arm and lumbar of the patient with a tape, a laser generation apparatus 4 engaged with a top face of the chamber 3, a liquid transfer tube 5 communicated with the chamber 3, a pressure reduction device 6 for reducing the pressured in the chamber 3 through the liquid transfer tube 5, a sensor 7 for measuring a glucose concentration of blood in the intercellular substance liquid sucked by the pressure reduction device 6, a body fluid collection receptacle 8 for accumulating the intercellular substance liquid transferred through the sensor, and a body fluid collection auxiliary sheet 9 to be in contact with the living body surface.

The chamber 3 is composed of transparent resin having a shape of an elliptical shell split into two parts along the major axis direction, and an interior of which is hollow to form a space for collecting body fluid and has a diameter of 2 cm and a height of 1 cm, for example. The bottom of the chamber 3 is opened as mentioned above so as to face a living body surface, and an entrance opening 3a for laser light to enter through is provided at the top. A dummy glass may be fitted in the entrance opening 3a for light incidence for air tightly separating the laser generation apparatus 4 from the chamber 3 to prevent the interior of laser generation apparatus 4 from being contaminated.

The laser generation apparatus 4 is constituted of a semiconductor laser 4b, a collimator lens 4c for converting the light from the semiconductor laser 4b into parallel light beam having a predetermined diameter, a driving circuit board 4d for driving the semiconductor laser 4b, and a case 4e connecting onto the chamber 3 for holding the above parts at the predetermined positions.

The body fluid collection auxiliary sheet 9 contains carbon black having a light-heat convergence function to absorb laser light and to convert the light to heat, and is in contact with the surface 2 of a living body and generates heat. When irradiated with the laser light, a minute hole is made in the surface 2 of the living body by heat.

As the semiconductor laser 4b, can be used an infrared semiconductor laser that emits infrared light, for example 780 nm, which is well absorbed in carbon black contained in the body fluid collection auxiliary sheet. At such wavelength, a low-cost semiconductor laser usually used in the CD-R drive can be utilized to lower the cost. In such wave length region, the absorbance in skin is very low, and it is difficult to make a minute hole the heat generated by the light directly absorbed in the skin.

To address this issue, a minute opening is made in a living body surface by the heat generated by near infrared laser light absorbed in the body fluid collection auxiliary sheet 9 having the above-mentioned photo-thermal convergence function. Thus, laser light that does not generate a desired heat when it is directly applied to a living body can be used.

On the driving circuit board 4d, a driving circuit for the semiconductor laser 4b, a circuit for controlling the entire apparatus, an operation section and a power source are installed, and the semiconductor laser 4b is driven so as to emit 20 irradiation pulses repeating on/off every 50 ms, for example. The irradiation with pulses of laser light makes a hole with a smaller diameter than one long time irradiation, although such effect depends on the absorption coefficient of the body fluid collection auxiliary sheet. The laser light from the semiconductor laser 4b is extended to a parallel light beam having a predetermined diameter by the collimator lens 4c and introduced into the chamber 3 through the entrance opening 3a.

The thickness of the body fluid collection auxiliary sheet is not more than 100 μm for example, and the diameter is about 1 cm so as to be enclosed in the chamber 3. The body fluid collection auxiliary sheet 9 is in contact with the living body 2, and then the chamber 3 is attached so as to surround the sheet. After that, the laser light is applied and the auxiliary sheet 9 converts the applied light into heat to form the hole in the living body surface 2. Accordingly, the minute hole can be surely formed by relatively small laser power. Thereafter, the intercellular substance liquid oozes out after removing the body collecting auxiliary sheet 9.

The minute hole having a diameter of about 100 μm and a depth of about 100 μm is formed in the living body surface 2 by the above-mentioned procedure. The diameter and the depth of the minute hole in the skin is such an extent that it removes the corneal layer and ecderon, and intercellular substance liquid of enough amount, about 1.4×10⁻³ mm³/sec for example, for measurement by the sensor 7 oozes out. When the depth exceeds 150 μm with an increased number of pulses applied, the hole reaches the corium and the blood can be collected from the capillary vessel. However, there are nerves there, and there is a possibility for a patient to feel pain. To the contrary, if the depth is less than 50 μm with insufficient number of pulses applied, only the corneal layer is removed, and the outer layer of skin, which is necessarily removed to cause intercellular substance liquid to ooze out, cannot be removed. Therefore, the depth of the minute hole is preferably from 50 to 150 μm, and most preferably 100 μm, to collect body fluid. Sufficient oozing out can be expected when the diameter is approximately the same as the depth. When the diameter is excessively large, the method becomes not low invasive for patients since the hole is clearly observed.

The pressure reduction device 6 as the sucking means is constituted of a suction pump, a power source, a controller circuit for them and an operation panel; the details of which are omitted. The pressure in the chamber 3 is reduced by the pressure reduction device 6, and the oozing out of body fluid from the minute hole is facilitated. The body fluid having oozed out is sucked and transferred through the liquid transfer tube 5 and the sensor 7 and then collected in the liquid collecting receptacle 8. The data measured by the sensor 7 are stored in the sensor 7 or transferred to a storage device by a communication means not shown in the drawing. The pressure reduction device 6 may provide a negative pressure required for collection, if desired, in response to an operation on the operation panel, or may operate, if desired, in conjunction with the driving circuit board 4d of the laser generating apparatus 4. Moreover, the pressure reduction device may be constituted so as to keep the required negative pressure in response to the detection result of a sensor provided in the liquid transfer tube 5.

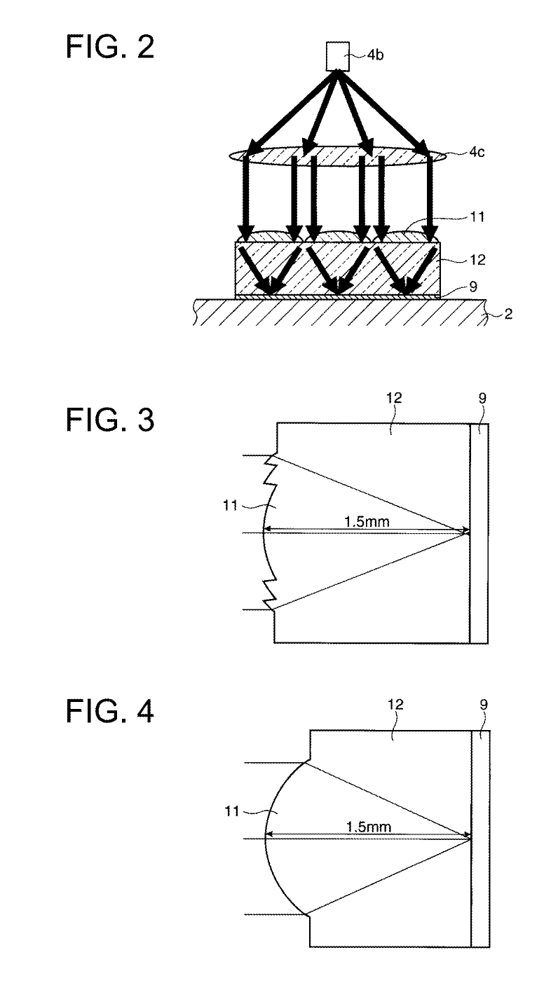

It is to be noticed in the body fluid collection device 1 of the embodiment of the invention that the body fluid collection auxiliary sheet 9 has the lenses 11 as the light convergence member for conversing the laser light applied on the surface opposite to the surface to be in contact with the living body surface 2 as shown in the enlarged drawing of FIG. 2. Moreover, a transparent member 12 is further placed between the lens 11 and the body fluid collection auxiliary sheet 9 so that the surface of the body fluid collection auxiliary sheet is positioned at the focal points of the lenses. Furthermore, the lenses 11 are arranged in an array and plural minute holes can be simultaneously formed.

As the lens 11, a Fresnel lens, a DOE (diffraction grating type lens), and a normal plastic lens are usable. FIG. 3 shows an example of the shape when the light convergence face side (the semiconductor 4b side) of the lens 11 is made in a Fresnel lens shape, and FIG. 14 shows an example of the shape when the light collecting face is made to be aspherical. The swell of the light convergence face can be reduced by employing a Fresnel lens shape, and the converged light spot can be made small by employing an aspherical shape so the efficiency of use light can be increased.

The lenses 11 and the transparent member 12 are preferably integrally molded, so that the production process of the body fluid collection auxiliary sheet 9 can be made simple and the cost can be lowered. The divergence property of laser of the light emitting point is generally different depending on direction; therefore, it is desirable for the shape of the lens 11 or the collimator lens 4c to be an anamorphic shape to meet the divergence property of the laser, in which anamorphic shape the focusing distance is different depending on the direction. By employing such constitution, the light from the semiconductor laser 4b can be converged in a smaller area so as to form a hole with high efficiency.

As described above, the body fluid collection auxiliary sheet 9, on which the lenses 11 are provided, has a part or a full of the light convergence function, therefore, if the laser generation apparatus 4 emits laser by accident, such a danger that the laser enters the patient's eye is low.

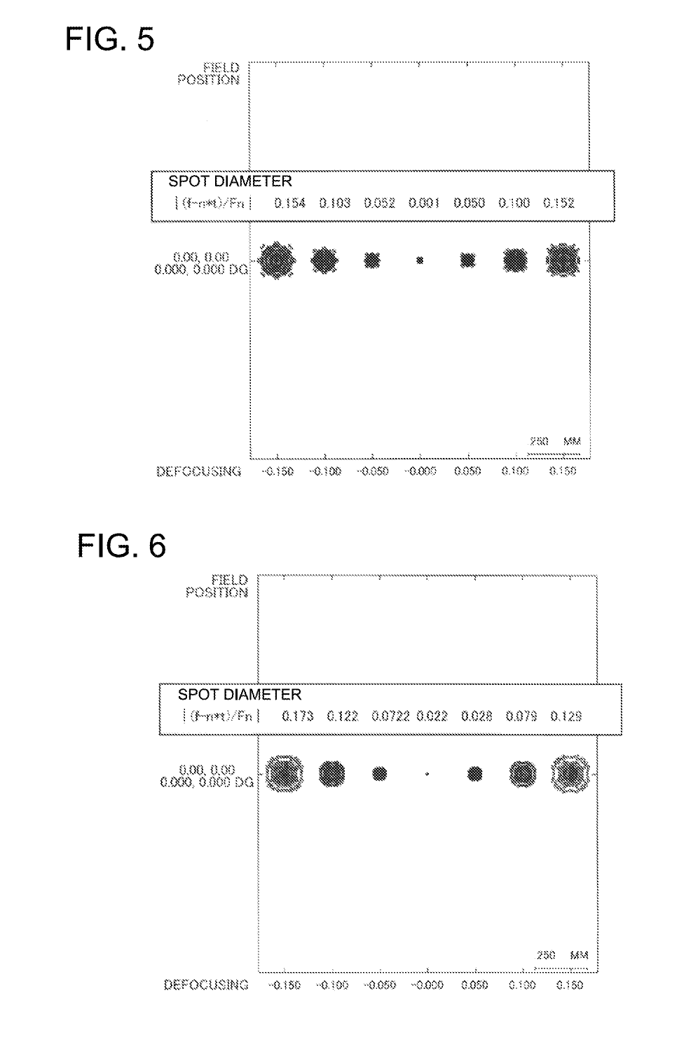

In FIGS. 5 and 6, the simulation results of the beam spot shape of the laser light with respect to the focusing error, when a Fresnel lens or an aspherical lens each having a focal length of 1.5 mm as shown in FIG. 3 or 4 is used. Parameters of the Fresnel lens are as follows:

| | |
|---|---|
| FNO | 0.98 |
| Focal length | 0.9810 |

| | Curvature | Surface | Refractive |
|---|---|---|---|
| | of radius | distance | index |
| Object | ∞ | ∞ | |
| surface | | | |
| First | 0.52045 | 1.5000 | 1.530480 |
| surface | | | |

| | | |
|---|---|---|
| Fresnel face coefficient | Largest sag amount | 0.1 mm |

| | | | | | |
|---|---|---|---|---|---|
| K: | 0.0 | | | | |
| A: | −1.6742E+00 | B: | 5.0099E+01 | C: | −7.0579E+02 |
| D: | 3.7112E+03 | E: | −4.0573E+03 | F: | −3.3949E+04 |
| G: | 1.3006E+05 | H: | −1.5251E+05 | | |

| | Curvature | Surface | Refractive |
|---|---|---|---|
| | of radius | distance | index |
| Second | ∞ | 0.000 | |
| surface | | | |
| Image | ∞ | | |
| surface | | | |

$\begin{matrix}z = \frac{{cr}^{2}}{1 + SQRT [ 1 - ( 1 + k ) {c}^{2} {r}^{2} ]} + {Qr}^{2} + {Ar}^{4} + {Br}^{6} + {Cr}^{8} + {Dr}^{10} + {Er}^{12} + \\ {Fr}^{14} + {Gr}^{16} + {Hr}^{18} + {Ir}^{20} + {Jr}^{22}\end{matrix}$

Parameters of the aspherical lens are as follows:

| | |
|---|---|
| FNO | 0.9943 |
| Focal length | 0.9943 |

| | Curvature | Surface | Refractive |
|---|---|---|---|
| | of radius | distance | index |
| Object | ∞ | ∞ | |
| First | 0.52747 | 1.5000 | 1.530480 |
| face | | | |

Non-spherical face coefficient

| | | | | | |
|---|---|---|---|---|---|
| K: | 0.000 | | | | |
| A: | 0.548746E+00 | B: | −0.285405E+02 | C: | 0.387242E+03 |
| D: | −0.280393E+04 | E: | 0.988947E+04 | F: | −0.139713E+05 |

| | Curvature | Surface | Refractive |
|---|---|---|---|
| | of radius | distance | index |
| Second | ∞ | 0.000 | |
| surface | | | |
| Image | ∞ | | |
| surface | | | |

$\begin{matrix}z = \frac{{ch}^{2}}{1 + SQRT [ 1 - ( 1 + k ) {c}^{2} {h}^{2} ]} + {Ah}^{4} + {Bh}^{6} + {Ch}^{8} + {Dh}^{10} + {Eh}^{12} + {Fh}^{14} + \\ {Gh}^{16} + {Hh}^{18} + {Jh}^{20}\end{matrix}$

Consequently, it is preferable that the lens 11 and the transparent member 12 satisfy the following expression (1), where the focal length (mm) of the lens 11 is f, the thickness (mm) of the transparent member 12 is t, the refractive index of the transparent member is n, and the full-open f number is Fn:

|(f−n·t)/Fn|≦0.2 (1)

When the spot diameter calculated by dividing the difference between the focal length f in air of the lens 11 and the effective length of optical pass n·t by the full-open f number Fn (the left side of the above expression) is exceeds 0.2, the focal point of the lens 11 is shifted from the surface position of the body fluid collection auxiliary sheet 9 so that the spot diameter is increased and the energy density is lowered. As a result, the energy necessary for the hole formation is increased or the opening diameter of minute hole is made lager and pain is caused. Such inconvenience can be prevented by making the above value to be 0.2 or less.

As mentioned above, the body fluid collecting auxiliary sheer 9 integrated with the lens for converging the applied laser light is used when making a minute hole by applying laser light to a living body surface in order to collect body fluid. Thus, the laser light can be converged at the specific point (the focal point of the lens 11), and the minute hole can be surely formed by relatively small laser power.

The distance between the lens 11 and the auxiliary sheet 9 can be precisely kept by further providing the transparent member 12 between the lens 11 and the auxiliary sheet 9 and arranging them in order of the auxiliary sheet 9, the transparent member 12 and the lens 11 from the side of the living body surface 2. Thus, the lens 11 executes its function as much as possible so that the minute hole can be formed with high efficiency. Moreover, the rigidity of the auxiliary sheet can be raised so that the minute hole can be surely formed by the generated heat by pressing the auxiliary sheet 9 to the living body surface 2. Furthermore, the plural minute holes can be simultaneously formed since the lenses 11 are arranged in the form of array, and the amount of the body fluid therefore can be raised.

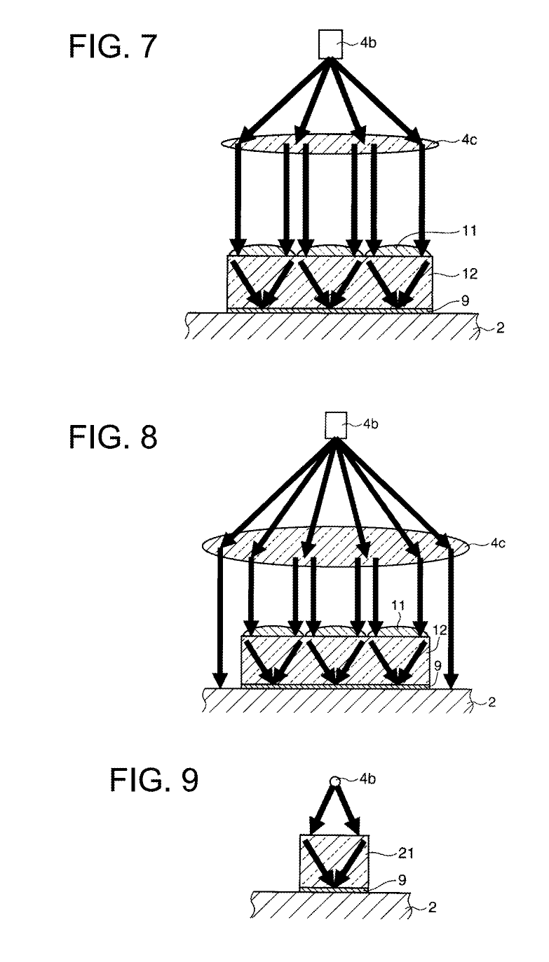

The laser light can be converged onto the body fluid collection auxiliary sheet 9 even when the nearing/leaving motion is caused, which means there is caused a deviation in the optical axis direction, as shown in FIG. 7, because the laser light from the semiconductor laser 4b is extended by the collimator lens 4c to be a parallel light having a predetermined beam diameter. The light can be converged onto the body fluid collection auxiliary sheet by making the diameter of the beam collimated by the collimator lens 4c to be larger than the body fluid collection auxiliary sheet 9 as shown in FIG. 8, even when a lateral motion or shifting in the direction of diameter of the collimator lens 4c is caused a little. In such case, the laser light directly applied to the living body surface 2 does not form any minute hole since such light is not converged and the energy thereof is thin. Thus, the influence of the shifting of position of the laser generation apparatus 4 and the body fluid collection auxiliary sheet 9 is reduced. As a result, the tolerance of attaching condition can be increased.

The optical distance between the collimator lens 4c and the lenses 11 can be made variable by making the case 4 to be extensible by utilizing the above-mentioned constitution, so that more suitable light convergence condition can be selected by controlling the light convergence condition. The sensor 7 is built in the device in the above example, but the structure may be modified such that the body fluid collection receptacle 8 can be detached to measure the body fluid collected in the receptacle 8 by a separate sensor. In such case, the burden on a patient can be further reduced since the sensor 7 does not need to be attached.

The body fluid collection auxiliary sheet 9 is removed after hole formation by laser light in the above example, but the intercellular substance liquid can be collected with the body fluid collection auxiliary sheet 9 being attached if a flow pass for the oozed intercellular substance liquid to flow through can be formed in the transparent member 12 by, for example, making a groove in the side, of the transparent member 12, facing the body fluid collection auxiliary sheet 9.

### Embodiment 2

FIG. 9 shows an enlarged view of a stacked construction of a body fluid collection auxiliary sheet 9 according to another embodiment of the invention. It is to be noted in this embodiment that a refractive index distribution type lens, in other words so called as GRIN lens 21, is stacked on the body fluid collection auxiliary sheet 9 as the light convergence member. The thickness is controlled such that the surface of the body fluid collection auxiliary sheet 9 is positioned at the focal point of the RGIN lens. Consequently, a planar light convergence member can be realized. Moreover, the function of the light convergence member and that of the transparent member can be performed by one sheet of lens.

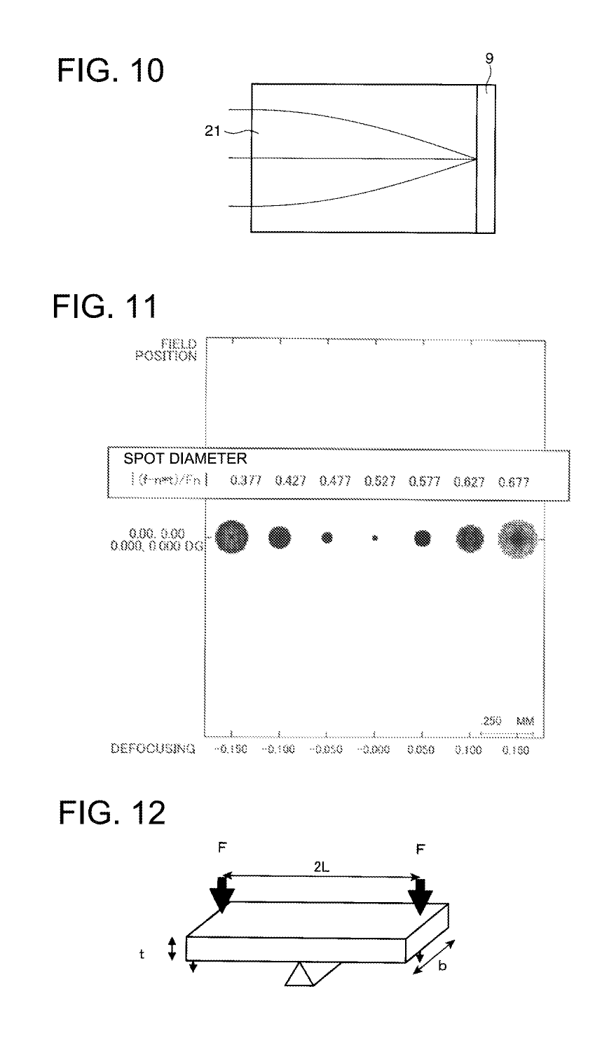

FIG. 10 shows an example of shape when the light convergence member to be stacked on the body fluid collection auxiliary sheet 9 is a GRIN lens 21. FIG. 11 shows the simulation results of the shape of beam spot of converged laser light with respect defocus amount when the GRIN lens as shown in FIG. 10 is used.

The lens parameters of the lens used in the simulation are as follows:

FNO 1.0
Focal length 0.6359

| | Curvature | Surface | Refractive |
|---|---|---|---|
| | of radius | distance | index |
| Object | ∞ | ∞ | |
| surface | | | |
| First | ∞ | 1.5000 | 1.530480 |
| surface | | | |

GRIN coefficient (Refer the following expression)

| | |
|---|---|
| n10 | −0.8085E+00 |
| n20 | 0.1446E−02 |
| n30 | 0.4462E−10 |
| n40 | 0.8548E−12 |

| | Curvature | Surface | Refractive |
|---|---|---|---|
| | of radius | distance | index |
| Object | ∞ | 0 | |
| surface | | | |
| First | ∞ | | |
| surface | | | |

n(r) = n₀₀ + n₁₀ r² + n₂₀ r⁴ + n₃₀ r⁶ + n₄₀ r⁸
r: Distance in the direction of light axis radius
n(r): Refractive index at the position of r of GRIN lens

The body fluid collection auxiliary sheet 9 may be formed to be larger than the chamber 3 so that the body fluid collection auxiliary sheet 9 can be pressed against the living body surface 2 by the entire circumference of the bottom surface of the chamber 3. Thus, the flatness of the living body surface 2 can be improved by pressing the body fluid collection auxiliary sheet 9. In such case, the size of GRIN lens 21 should be made so as to be surely included in the chamber 3.

In this case, it is preferred that the following conditional expression is satisfied where the distance between the supporting points on the occasion of pressing the body fluid collection auxiliary sheet 9 against the living body surface 2 is 2·L (mm), the open f number of the lens is Fn, the thickness is t (mm) and the Young's modulus is E (kg/m²).

0.01<L²/(Fn·E·t³)<0.1 (2)

FIG. 12 shows a modeled shape variation of a plate-shaped body. The positional deviation 8 of each end is expressed by the following expression where the fulcrum is assumed to be placed at the center, the load applied to each end is F (kg), the Young's modulus is E (kg/mm²), the length of the plate-shaped body is 2-L (mm), the thickness is t (mm) and the width is b (mm).

δ=(4·F·L³)/(F·b·t³)

When the plate-shaped body is a square, and 2·L=b and F=0.5 kg, the expression is as follows.

δ=(2.0.5·L³)/(E·2·L·t³)=(0.5·L²)/(E·t³)

Consequently, when δ is lower than the lower limit of expression (2), the thickness of the transparent member becomes too thick, thereby increasing the cost of the body fluid collection auxiliary sheet 9. On the other hand, when the value exceeds the upper limit, the body fluid collection auxiliary sheet 9 is excessively deformed on the occasion of being pressed against the living body surface 2, and the focal point of the lens 11 and 21 is shifted from the surface of the body fluid collection auxiliary sheet 9. As a result, the spot diameter is increased and the energy density is lowered so that the energy necessary for forming the hole is raised or the diameter of the formed minute hole is increased, thereby causing pain. Accordingly, such troubles can be prevented by satisfying the relation of the expression 2.

According to the invention, the light convergence member for converging the applied laser light is provided on one side of the body fluid collection auxiliary sheet, the other side of which auxiliary sheet is stuck to the living body surface. The body fluid collection auxiliary sheet 9 is used at the time of forming the minute hole by applying laser light to the living body surface to collecting body fluid, such as blood and intercellular substance liquid, oozing through the minute hole.

According to the above structure, provided are a body fluid collection auxiliary sheet by which the laser light can be preciously converged so that the minute hole can be surely formed by relatively small laser power, and the body fluid collection device using the body fluid collection auxiliary sheet.

The detailed constitution and operation of the body fluid collection auxiliary sheet and of the body fluid collection device using the same both according to the invention may be suitably modified without departing from the spirit of the invention.

1.-9. (canceled)

10. A body fluid collection auxiliary member for collecting body fluid, the auxiliary member, comprising:
a contact portion for contacting with a living body surface; and
a light convergence member provided facing the contact portion,
wherein laser light applied to the light convergence member is converged by the light convergence member onto a vicinity of the contact portion to make a minute hole in the body surface through which body fluid oozes out.

11. The body fluid collection auxiliary member of claim 10, wherein the contact portion has a photothermal convergence function for absorbing the laser light and converting the laser light into heat, and the minute hole is made by the converted heat.

12. The body fluid collection auxiliary member of claim 10, comprising:
a transparent member provided between the light convergence member and the contact portion,
wherein the light convergence member is a convergence lens, and a focal point of the convergence lens is located in a vicinity of the contact portion.

13. The body fluid collection auxiliary member of claim 12, comprising:
a plurality of the convergence lenses provided in an array so as to simultaneously make a plurality of the minute holes in the living body surface.

14. The body fluid collection auxiliary member of claim 12, wherein the convergence lens is a graded index lens, and the auxiliary member is configured such that the surface of the contact portion is positioned at a vicinity of a focal point of the graded index lens.

15. The body fluid collection auxiliary member of claim 12, wherein the following conditional relationship is satisfied:

|(f−n·t)/Fn|≦0.2
where:
f (mm) is a focal length of the lens in air;
t (mm) is a thickness of the transparent member;
n is a refractive index of the transparent member; and
Fn is an open F number of the lens.

16. The body fluid collection auxiliary member of claim 11, wherein the auxiliary member is configured to be pressed via a plural points or an annular portion of the auxiliary member against the living body surface, and the following conditional relationship is satisfied:
0.01<L²/(Fn·E·t³)<0.1
where:
(mm) is a distance between portions via which the auxiliary member is pressed against the living body surface;
Fn is an open F number of the convergence lens;
t is a thickness of the auxiliary member;
E is a Young's modulus of the auxiliary member.

17. A body fluid collection device, comprising:
a chamber for defining a space for collecting body fluid, the chamber including a top surface having a first opening and including a bottom surface having a second opening; and
a laser generation device provided on the chamber so as to supply laser light into the chamber through the first opening, the laser generation device including:
a semiconductor laser for emitting the laser light; and
a collimator lens for converting the laser light from the semiconductor laser into a parallel light having a predetermined beam diameter to supply the converted laser light into the chamber through the first opening,
wherein, when the body fluid collection device is positioned, in contact with the living body surface, such that the second opening surrounds at least a part of the body fluid collection auxiliary member of claim 10 placed on the living body surface, the laser light supplied into the chamber is applied to the light convergence member.

18. The body fluid collection device of claim 17, further comprising:
a suction member provided in communication with the chamber to reduce a pressure in the chamber so as to facilitate the body fluid to ooze out, and to suction the body fluid which has oozed out.

19. The body fluid collection device of claim 17, wherein the body fluid collection auxiliary member is pressed against the living body surface by the bottom surface of the chamber via an annular portion of the auxiliary member.

20. The body fluid collection auxiliary member of claim 12, wherein the transparent member includes a flow pass through which the body fluid flows.
